(19) United States
(12) Patent Application Publication
(10) Pub. No.: US 2009/0128145 A1
(43) Pub. Date: May 21, 2009

(54) Apparatus and method for obtaining magnetic resonance imaging or spectroscopy data from live tissue samples

(76) Inventors: Timothy M. Shepherd, Jacksonville, FL (US); Bjorn Scheffler, Gainesville, FL (US); Stephen J. Blackband, Gainesville, FL (US)

Correspondence Address: Beusse Wolter Sanks Mora & Maire, 390 N. ORANGE AVENUE, SUITE 2500, ORLANDO, FL, 32801, US

(21) Appl. No.: 11/914,792

(22) PCT Filed: May 18, 2006
(86) PCT No.: PCT/US06/19048

§ 371 Date: Jun. 26, 2008

Related U.S. Application Data
(60) Provisional application No. 60/682,625, filed on May 19, 2005.

(51) Int. Cl. G01V 3/14; C12N 5/02

(52) U.S. Cl. 324/307; 435/395

(57) ABSTRACT

Disclosed herein is a system useful for obtaining magnetic resonance imaging data of longterm tissue slice cultures that typically survive well beyond 12 hours after tissue procurement. The system comprises a first support member comprising a perimeter defining a first interior space and a second support member. The first and second support members are configured such that they can secure a substrate having a live tissue sample disposed thereon when said first and second support members are brought together. Further, the system comprises a chamber into which the first and second support members, when brought together, may be disposed. Also disclosed herein is a method of producing magnetic resonance imaging data from a live tissue sample, said method comprising obtaining a tissue sample comprising live cells, culturing said tissue sample under conditions to keep cells of said tissue alive for at least 12 hours, and subjecting said tissue sample to magnetic resonance imaging.

### RELATED APPLICATIONS

This application claims priority to U.S. Ser. No. 60/682,625 filed May 19, 2005, which is incorporated herein in its entirety by reference.

### BACKGROUND

Magnetic Resonance Imaging (MRI) has revolutionized clinical medicine over the past 25 years by providing detailed anatomical information and surrogate markers of disease non-invasively for many different organs in the human body, especially the brain. However, many current questions in magnetic resonance imaging research, such as understanding the biophysical basis for water diffusion in nervous tissue (1), may require experiments in high-field magnets with powerful imaging gradients and long acquisition times to be resolved. This knowledge could improve the sensitivity and specificity of different MRI contrast mechanisms for different human disease processes (e.g. ischemic stroke) and thus, facilitate clinical decision-making. Technologies that may incorporate MRI for stem cell and molecular imaging may also require similar conditions for their initial development prior to in vivo experiments in animal or human subjects. Experiments that meet these criteria require alternative tissue models because the experiments are presently difficult, if not impossible, to conduct using animal or human subjects because of limitations in subject tolerance (e.g. for experiment duration and specific absorption rate of radiofrequency power). Novel technological developments, such as MRI-based molecular imaging and stem cell imaging, also may benefit from simplified tissue models imaged at high fields that provide near-complete experimental control over the tissue environment.

One solution to these challenges is to conduct MRI experiments on viable, perfused brain slices acutely-prepared from animals or human surgical biopsy specimens (2,3). These brain slices retain the 3-dimensional cytoarchitecture of in vivo nervous tissue, but tolerate relatively long imaging protocols in high-field, narrow-bore magnets. In addition, brain slices no longer have a blood-brain-barrier, which gives complete experimental control over the extracellular environment of the tissue. These properties have enabled studies of the water diffusion changes that follow pharmacological or osmotic perturbations to rat and human hippocampal slices (4, 5 & 6). Recently, these methods also facilitated the measurement of the mean intracellular residence time in rat and human cortical slices (7), which has proven difficult to measure accurately in vivo.

Acutely-prepared brain slices have some limitations that have prevented their more widespread use as a tissue substrate for MRI experiments and technology development. Acutely-prepared slices appear viable by electrophysiology, histology and diffusion MRI (2) for only 10-12 hours when imaged at room temperature (20° C.) with intermittent perfusion. This relative hypothermia is neuroprotective and facilitates longer experiment times, but also may blunt normal and pathological responses of the nervous tissue to experimental perturbation. Further, proteolytic enzyme cascades may be activated by the slice procurement process despite normal-appearing cell and tissue morphology in the slices. The slices also have approximately 50-micron deep zones of surface tissue injured from slicing during the procurement process. These zones must be excluded from MRI analysis and necessitate that acutely-prepared slices are several hundred microns thick so that healthy tissue can be imaged. This thickness in turn makes it difficult to use correlative techniques, like confocal microscopy, on live slices. These limitations suggest that MRI of acutely-prepared brain slices are best used only for studies of unperturbed tissue or tissue exposed to acute pharmacological perturbations.

### BRIEF DESCRIPTION OF THE DRAWINGS

FIG. 1 shows a top view of an embodiment useful for obtaining MRI data of cultured tissue samples.

FIG. 2 shows 78-μm in-plane resolution diffusion-weighted images (b=2028 s/mm²) of 4 axially-cut, P9 rat hippocampal slices after incubation for 2 weeks [100-μm image thickness, TR/TE=1500/23.3 ms, 64 averages, scan time=3.5 hrs]. Despite the limited thickness of these hippocampal slice cultures (˜150 μm), there is sufficient MRI signal to clearly demonstrate the laminar anatomy of the hippocampus (CA), dentate gyrus (DG), subiculum (SUB), entorhinal (ENT) and temporal cortex (TC). The region of decreased signal within the slices (arrow) atrophies after slice procurement. Microbubbles noted in the images (arrowhead) were trapped underneath the cell culture membrane inserts during chamber assembly.

FIG. 3 shows a normalized log-linear plot of diffusion-weighted signal attenuation curves for rat hippocampal slice cultures at three different diffusion times (Td) [mean±SD, 11 slices]. Dispersion of the three curves indicates that signal attenuation is affected by restriction and/or exchange at the diffusion times used (10-35 ms). This pattern has been observed in other nervous tissue (7) and suggests the slice cultures maintain a 3-dimensional tissue microstructure that is a valid model for in vivo brain.

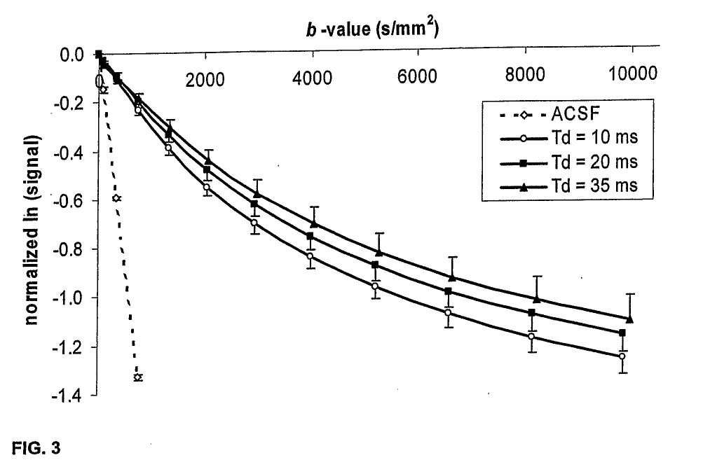

FIG. 4 shows a gradient echo image of a rat hippocampal slice culture (39-μm in-plane resolution, thickness=80 μm, TR/TE=185/12.5 ms, flip angle=30°, time=25 min). Gradient echo contrast distinguishes several regions of hippocampal anatomy (TC=temporal cortex, ENT=entorhinal cortex, SUB=subiculum, DG=dentate gyrus, CA=cornu Ammonis) and could be used to track stem cells or molecular imaging agents labeled with T₁ or T₂ contrast agents. The image includes the free edges of the culture membrane (black arrows) and the top Delrin bracket (white arrow—see FIG. 1 schematic). Microbubbles of air can form under the surface of the cut culture membrane (e.g. white arrowheads).

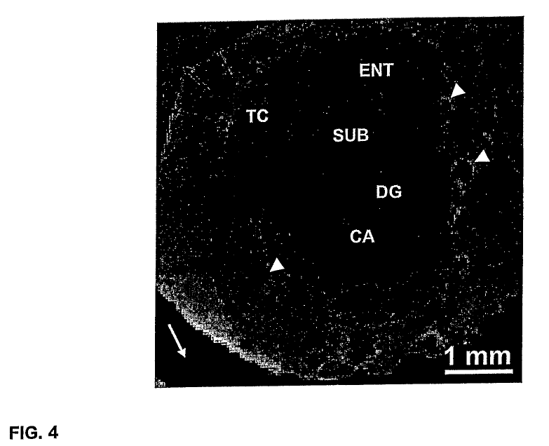

FIG. 5 The viability of slice cultures under predetermined conditions was qualitatively assessed by immersing slices cultured for 15 days in culture media for up to 8 hours at 20° C. or 35° C. (A). Phase-contrast microscopy of cultured hippocampal slices after 4 hours immersion did not indicate structural pathology (B) [white box indicates field-of-view for panels C-E]. Further, NeuN and GFAP fluorescent immunostaining indicate relative stability for more than 4 hours after immersion (C-D), but demonstrate some neuronal soma shrinkage and increased GFAP immunoreactivity consistent with reactive gliosis after 8 hours incubation (E).

FIG. 6 shows a top view of several alternate bottom insert embodiment configurations (a-e) which may facilitate perfusion of cultured tissue slices in a cylindrical tube, but redirect flow away from the surface of the tissue culture.

FIG. 7 shows a perspective view of an expanded apparatus embodiment for MR imaging of slice cultures, which may be assembled inside a 10-mm NMR tube filled with culture media. The arrows indicate the flow of media if tissue perfusion is desired.

FIG. 8 shows top-view of a disassembled non-cylindrical embodiment of the invention that may be used for example with custom-built coils that better optimize SNR for the sample as compared to the standard magnet radiofrequency coils.

FIG. 9 shows an embodiment that is a one-piece construction that may be used for culturing tissue and subsequently placed in a glass NMR tube or customized chamber for MRI of the cultured tissue attached. FIG. 9 (a) is a top view of the embodiment. FIGS. 9 (b)(1) and (c)(1) are top views of the embodiment wherein transverse lines B and C represent orthogonal cross-sections of this piece. FIGS'. (b)(2) and (c)(2) represent the respective cross section. This piece includes one or more distinguishing features; a low profile (˜500 μm) so that many slice cultures could be imaged simultaneously, a diameter (*)<1-2 mm more than the greatest dimension of the tissue slice (e.g. 6-7 mm for hippocampal slice cultures), and would have large gaps in the culture membrane for the free passage of culture media during MRI setup and perfusion. Because these pieces would have low vertical profiles, many such pieces could be used in an NMR tube to simultaneously acquire MRI data from 20+ cultured tissue slices simultaneously. A perfusion line also could be placed through the large gaps in the culture membrane of individual pieces to perfuse culture media through the samples during an MRI acquisition (with a similar flow pattern to that shown in FIG. 7, but without the bottom anchor). FIG. 9(d) relates to an alternative embodiment of a one-piece construction. FIG. 9(e) relates to another alternative embodiment.

### DEFINITIONS

It is important for an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding of the invention as disclosed and claimed herein, the following definitions are provided.

Magnetic Resonance Imaging (MRI) uses a strong static magnetic field, B₀, to polarize nuclear spins in a sample. Usually the hydrogen nuclei on water are examined since these are the most abundant in tissue (and thus give the most signal), but other nuclei are not excluded. These spins can then be perturbed from equilibrium by applying a second orthogonal magnetic field, B₁, usually referred to as the radiofrequency (rf) pulse. This pulse is applied using an rf coil placed around the sample. After excitation, the precessing hydrogen nuclei induce a signal in a detector rf coil. That signal can then be spatially encoded in some way to produce what is termed the MR image. The most common method for spatial encoding is to apply three orthogonal linear magnetic field gradients in various combinations. There are many so-called pulse sequences that can be used to acquire a variety of image types. All use the gradients to encode the signal as a function of space, so generating an image. The amount of signal (or signal-to-noise ratio —SNR) available per unit time depends on many factors, but most importantly the magnet field strength, and the quality and size of the radiofrequency coil size relative to the sample. This SNR can then be used for high spatial or temporal resolution or to acquire different contrast images from the same sample. For purposes of this application, magnetic resonance imaging or MRI is used in a broad sense to also refer to magnetic resonance spectroscopy, unless otherwise indicated. Alternative nuclei (e.g., fluorine, sodium, C¹³) can also be studied with nuclear resonance.

The term “tissue slice” as used herein refers to a sample of tissue removed from a tissue generated in vivo and portioned such that the three dimensional cyto-architecture of the tissue sample is preserved. In embodiments where the ability to conduct confocal microscopy on the tissue is desired, the tissue slice preferably comprises a generally uniform thickness. Slice culture thickness may range anywhere from 25 to 1000 micrometers. Preferably, certain characteristics of the organ that the tissue slice was removed from are maintained, e.g. preservation of organotypic neural circuitries.

Culture conditions—Two fundamentally different tissue slice paradigms provide the basis for modern biomedical experimentation. One is the immediate evaluation of derived tissue in very simple buffer- and salt-solutions, such as the common use of artificial cerebrospinal fluid (ACSF) for sliced brain tissue. In these environments, however, structure and function of “acutely isolated slices” will rapidly decline within hours after derivation. Tissue slices can, on the other hand, be cultured using more sophisticated equipment (cell culture incubators and dedicated plastic ware) and special nourishing media tailored for the particular needs of the extracted tissue. Culturing a tissue slice can maintain the vitality of the tissue for prolonged periods of time (several weeks in general). Commonly used methods for the maintenance of “long-term slice cultures” are the roller tube technique, and the membrane technique, the latter representing the basis of studies in this proposal. Cultured tissue is also maintained on a porous membrane in an interphase environment provided by the incubator (humidified 5% CO₂ atmosphere at 35° Celsius), and nourishing media underneath the membrane (8, 9).

The term “substrate” as used herein refers to any surface upon which cells or tissue are able to grow and preferably to which they can attach, and whose interference with the MRI signal from magnetic susceptibility artifacts is minimal. In no way intended to be limiting, examples of substrates that may be used in accordance with the subject invention, include but are not limited to, culture membranes such as, PTFE membrane, mixed cellulose esters membrane, and polycarbonate membrane, polyethylene terephthalate surfaces, Millicell-CM® Low Height Culture Plate inserts (MILLIPORE) or polystyrene culture plate surface.

The term “not proximate to tissue on substrate” in the context of allowing fluid to pass by the substrate means that the predominance of fluid crossing the plane of the substrate does not occur beneath or immediately contiguous to the tissue.

### DETAILED DESCRIPTION

The inventors have discovered that accurate MRI data can be reproducibly obtained from tissue slices that have been explanted and cultured for twelve hours or more. Thus, according to one embodiment, the subject invention pertains to a method of producing magnetic resonance imaging data from a live tissue sample, said method comprising obtaining a tissue sample comprising live cells, culturing said tissue sample under conditions to keep cells of said tissue alive for at least 12 hours, and subjecting said tissue sample to magnetic resonance imaging.

The inventors have found that the method of producing magnetic resonance imaging data of cultured tissue slices possesses several advantages over the prior art employment of acutely-prepared tissue slices. Acutely-prepared slices are viable for electrophysiology, histology and diffusion MRI for only about 10 hours when imaged at room temperature (20° C.) with periodic perfusion. Furthermore, the need to keep the acutely prepared slices at such low temperature for neuroprotection and facilitation of longer experimental times inhibits normal and pathological responses of the nervous tissue to experimental perturbation. Further, proteolytic enzyme cascades are activated by the slice procurement process despite normal-appearing cell and tissue morphology in the slices. The slices also have approximately 50-micron deep zones of surface tissue injured from slicing during the procurement process. These zones must be excluded from MRI analysis and necessitate that acutely-prepared slices are several hundred microns thick, which in turn makes it difficult to use correlative techniques, like confocal microscopy, on acutely-prepared live slices. In contrast, MRI of cultured tissue slices overcomes these problems. The culturing of tissue slices allows them to remain viable for more than 10-12 hours, and they can remain viable for more than 18 hrs, 24 hrs, 36 hrs, 48 hrs or several days or even weeks. Also, the slices may be studied at temperatures more closely emulating in vivo conditions. Further, the necessity to obtain thick slices is obviated, as thinner slices may be explanted and stabilized while maintaining the three-dimensional cytoarchitecture or organotypic structure. This in turn allows for important correlative studies on the tissue slices to be conducted such as confocal microscopy. The cultured tissues also are frequently attached to a membrane insert that facilitates MRI geometry during experiment setup and prevents tissue movement during the MRI acquisitions.

So long as a tissue sample may be explanted and kept viable in culture for 12 hours or more, the tissue may be obtained from any part of the body of human and nonhuman animals. Tissue that may be studied according to the methods of embodiments herein include, but is not limited to, central nervous system tissue, cardiac tissue, vasculature tissue, skeletal muscle tissue, gastrointestinal tissue, glandular tissue, liver tissue, spleen tissue, connective tissue, or bone tissue. In a specific embodiment, the tissue that may be studied is brain tissue. In alternative embodiments, the tissue sample is derived from cells in a suspension which have been attached to the substrate and grown into a mass of contiguous or interactive cells.

According to another embodiment, the subject invention pertains to a system useful for obtaining magnetic resonance imaging data of a live tissue sample. The system comprises a first support member comprising a perimeter defining a first interior space and a second support member. The first and second support members are configured such that they can secure a substrate having a live tissue sample disposed thereon when said first and second support members are brought together. Further, the system comprises a chamber into which the first and second support members, when brought together, may be disposed. The chamber is preferably comprised of a material suitable for obtaining magnetic resonance imaging data from said live tissue sample when contained within said chamber. The chamber may take a multitude of different shapes and dimensions.

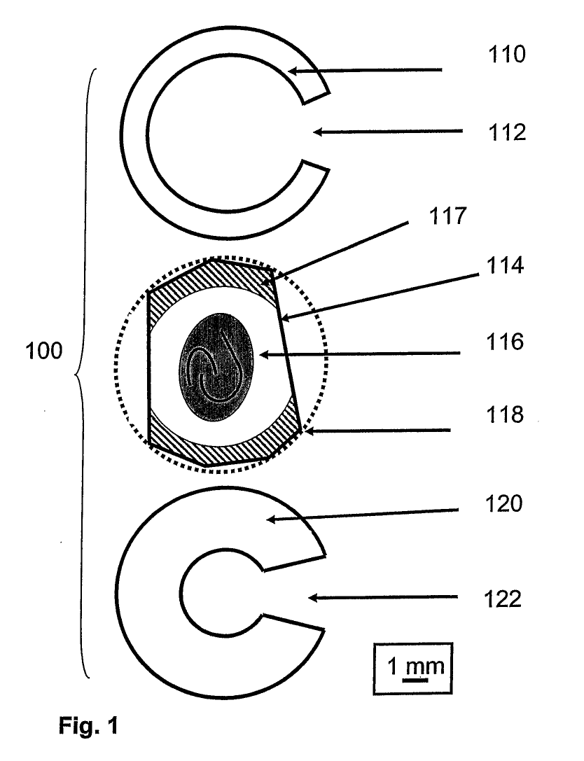

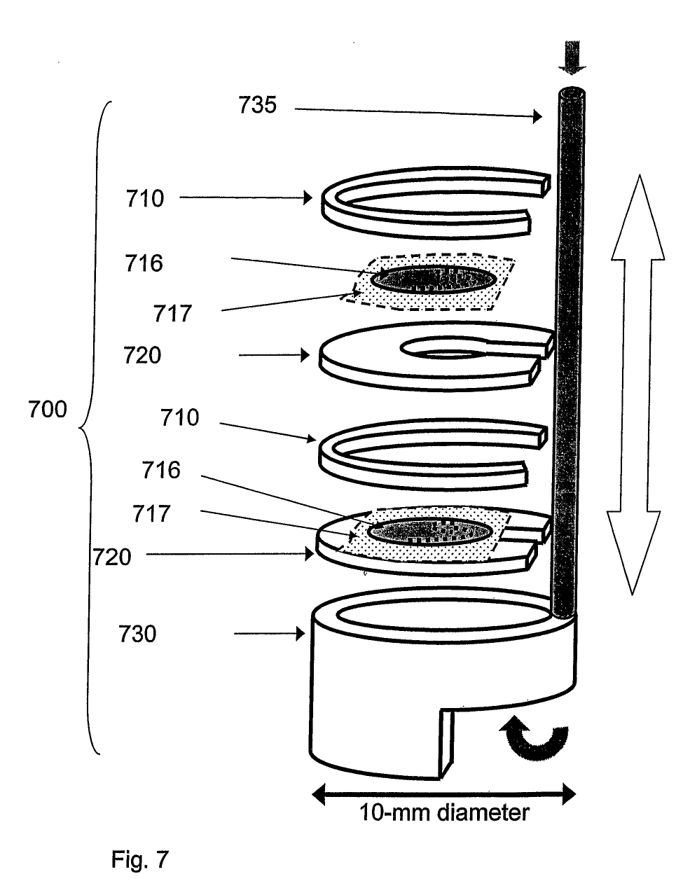

FIG. 1 represents an embodiment comprising an assembly 100 for securing a cultured tissue slice 116 grown on a culture membrane 118 or other substrate for use in an MRI chamber. The assembly 100 comprises a top bracket 110 that comprises a notch 112 and a bottom bracket 120 comprising a notch 122. The culture membrane 117 in this embodiment happens to be cut so that it has free edges 114 and pressed edges 118. The pressed edges 118 are secured by the pressing caused by the top and bottom brackets 110, 120 being brought together. The culture membrane 117 may be obtained from cutting away a membrane attached to a conventional cell culture insert and trimmed to fit inside a 10-mm NMR tube (inner diameter ˜9 mm). The notches 112,122 in the top and bottom brackets 110, 120 are used for passage of an anchor tube (see FIG. 7), which can then be used to remove the sample after imaging is complete.

FIG. 7. represents an embodiment of a disassembled apparatus 700 for obtaining magnetic resonance imaging data of live tissue. The apparatus 700 is designed for positioning in an MRI chamber (not shown). The apparatus 700 is designed for holding a plurality of slice cultures 716 grown on a membrane 717 or other suitable substrate. A top bracket 710 and bottom bracket 720 with a membrane 717 holding a slice culture 716 are brought together similar as described for FIG. 1. Two or more series of assemblies comprising top and bottom brackets 710, 720 with membrane 717 held therein between may be stacked one on top of the other. The top bracket 710 creates a space between the slice culture 716 and the next succeeding bottom bracket 720 positioned above the top bracket 710. This prevents the slice culture 716 from being crushed as the series of assemblies are stacked. The series of assemblies are stacked on top of an anchor piece 730. Attached to the anchor piece is a hollow tube 735. The tube 735 and anchor piece 730 assist in the inserting and removing of the stacked plurality of assemblies in and out of a MRI chamber. This may be accomplished with some other structure attached to the anchor 730. The tube 735 may be hollow and associated with the anchor 730 in such a way so as to deliver fluids to the stacked assemblies to perfuse the cultured tissue 716. The top brackets 710 and bottom brackets 720 comprise a notch 722 so as to allow space for the tube 735 to pass. Furthermore, the notch 722 comprises more space than needed by the tube which allows fluid to pass by the membrane 717 and tissue culture 716 so as to not create fluid pressure that might shear the tissue 716 from the membrane 717.

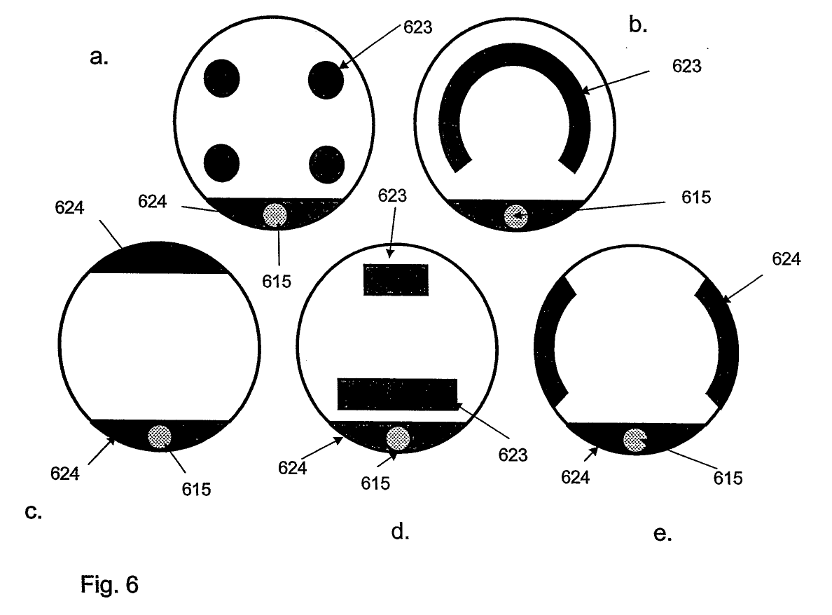

FIG. 6 (a)-(e) represent different configurations of a bottom bracket that may be substituted with the bottom bracket 120 shown in FIG. 1. The bracket comprises openings 623 or gaps 624 that facilitate perfusion of cultured tissue slices in a cylindrical tube, but redirect flow away from the surface of the tissue culture. A cross section of an inflow tube or anchor 615 is shown as being positioned in the gaps 624.

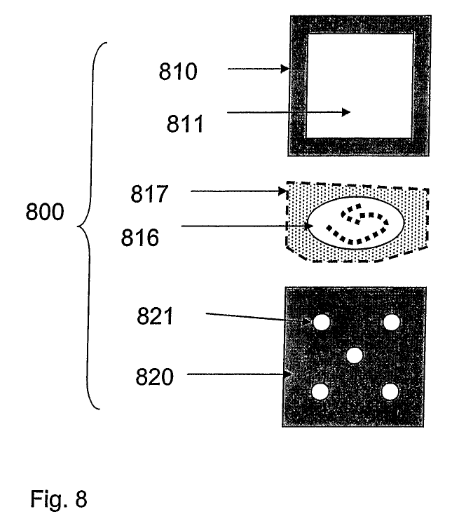

It may be preferable that the chamber be constructed as a cylindrical device and placed with its main axis parallel to that of the applied magnetic field B₀. In this way conventional rf coils can be used and the system has optimal geometry for magnetic field shimming, thus minimizing image distortions and maximizing the SNR. This is not however fundamentally necessary and good data could be obtained with chambers of alternate geometries, with or without custom built rf coils. FIG. 8 shows and alternative assembly embodiment 800 comprising a squarely shaped top bracket 810 and a squarely shaped bottom bracket 820. A tissue 816 is disposed on a membrane 817 which is sandwiched in between the top and bottom brackets 810, 820. Typically, the space 811 created by the top bracket 810 is larger than the cross-sectional area of the cultured tissue 816 to be imaged and allows space for the passage of tissue culture media (as perfusate). The bottom bracket 820 comprises holes 821 for passage of fluid. This arrangement also could be oriented orthogonal to the horizontal plane for particular rf coil designs so long as the “top” bracket 810 substantially surrounds the tissue.

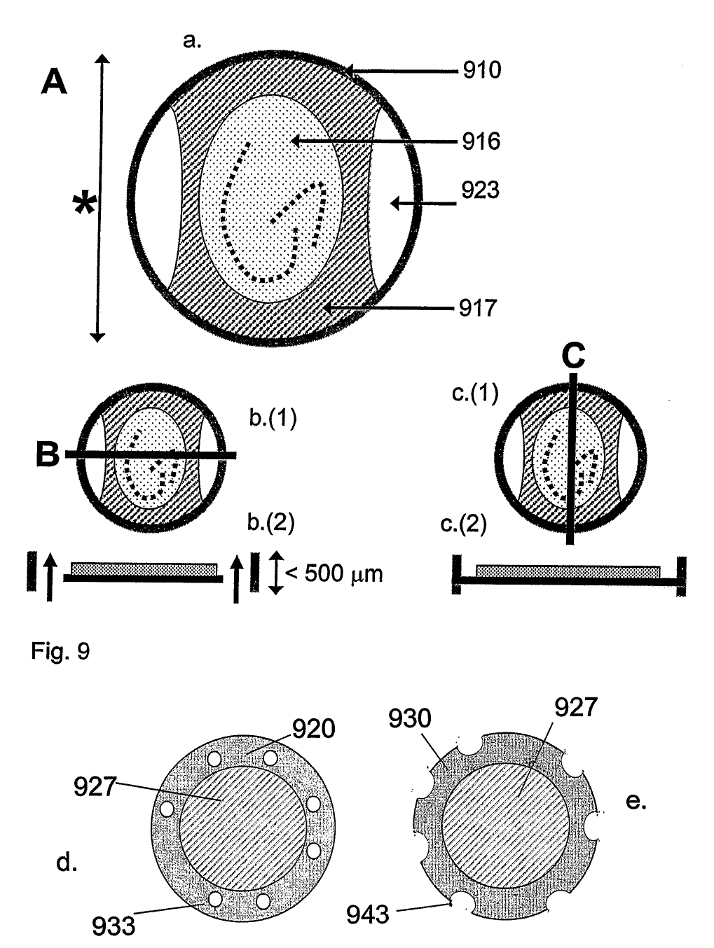

Alternatively, the device for imaging slice cultures could be a combination of the tissue culture membrane insert and top and bottom brackets into one manufactured piece that could be used for surface-interface culture of the tissue and subsequently placed into an NMR tube or custom-built chamber for obtaining MRI data. FIG. 9 (a) shows a top view of a one-piece design embodiment 900 for use in culturing tissue for placement in an MRI chamber. This one piece embodiment 900 comprises a peripheral support structure 910 integrated with or attached to a culture membrane 917 onto which a cultured tissue slice 916 is disposed. Typically, the support structure comprises a low profile (typically, less than 500 μm in height) which would allow the stacking of multiple slice cultures to be imaged simultaneously. The one-piece embodiment 900 comprises one or more openings 923 provided away from the cultured tissue 916 so as to allow the passage of culture media both during perfusion and also during setup (in the inventors' experience, the tissue will be sheared frequently from the membrane insert during setup without this feature). These 3 key features differ significantly from current commercial products used solely for culturing tissue slices (e.g. Millicell-CM® Low Height Culture Plate inserts by Millipore). FIG. 9 (b)(2) shows a side-view cross section of the one-piece embodiment 900 along plane B of FIG. 9 (b)(1). FIG. 9 (c)(2) shows a side view cross section of the one-piece embodiment 900 along plane C of FIG. 9 (c)(1). A portion 925 of the support structure 910 extends slightly below the membrane 917 to provide additional space for tissue culture media during the pre-MRI surface-interface culture conditions required to maintain tissue viability.

The openings 923 may be located interior to the inside surface of a support structure 917, such as shown in FIG. 9(a). Alternatively, as shown in FIG. 9(d), openings 933 are defined in the support structure 927 itself, such as by holes provided in the support structure. Further, as shown in FIG. 9(e), openings 943 are defined contiguous to the outer surface of a support structure 930. In another embodiment (not shown), the present invention is directed to a MRI chamber configured for receiving a one-piece scaffold without openings, wherein at least one channel for passing fluid across the plane of the substrate is defined in the wall of the MRI chamber.

Furthermore, it is important that the tissue remain relatively still and in place during the MRI process to prevent movement artifacts in the MRI data. The structural support members and membrane may be in certain embodiments relatively small and difficult to manipulate, and insert and remove from the chamber. If fluid is in the chamber, or will be added to the chamber after the support members and substrate are added, the fluid may disrupt the tissue sample, causing damage to the tissue, or where the tissue is attached to the substrate, undesired detachment from the substrate. Accordingly, it is important in certain embodiments that the support members and substrate are secured in the chamber but that movement of fluid does not unnecessarily disrupt the tissue on the membrane. Accordingly, either of the support members, or the substrate, or some combination thereof, are configured to allow fluid to predominantly cross a plane of the substrate without disrupting the tissue sample. The substrate may comprise small pores that allow fluid to cross a plane of the substrate, but fluid will predominantly pass the plane of the substrate at one or more locations that are positioned so as to not disrupt the tissue by the fluid dynamic forces. The fluid may pass by one or more portals in the first or second support members or substrate, or combination thereof. The portal(s) may be the form of notches, pores, channels, etc, defined on one or more of these components.

According to another embodiment, the subject invention is directed to a novel tissue slice culturing scaffold. The scaffold comprises a substrate onto which a tissue slice may be disposed and comprises a perimeter around the area where a tissue slice is disposed. Integrated with or attached to at least a portion of the perimeter is at least one support structure. Then at least one support structure is configured to engage an inner wall of a chamber to thereby secure said substrate in said chamber. Furthermore, the scaffold may be configured to have opening(s) to allow fluid to predominantly cross a plane of said substrate at at least one location that is not proximate to a tissue when disposed on said substrate. The term “not proximate” means that the place at which fluid crosses the plane of the substrate does not overlap the area where a tissue sample is disposed so that the tissue sample is not disrupted during assembly of the scaffold into an MRI chamber. In an alternative embodiment, the one-piece scaffold does not have a portal for passing fluid, but nonetheless has a low profile support structure of 5 mm or less, 2 mm or less, 1 mm or less, 500 microns or less, or 250 microns or less. This one piece scaffold could be used for imaging of tissue culture cells and/or tissue using techniques other than MRI, such as, for example, two-photon microscopy.

Those skilled in the art will also appreciate that certain embodiments herein could be adapted for nonstandard radiofrequency coil designs that are better suited/optimized to the size and thickness of the sample in order to maximize the signal-to-noise ratio (SNR) obtained from MRI of the tissue.

In another embodiment, the subject invention pertains to a method that utilizes an arrangement such as that shown in FIG. 1 but where no membrane is used. Tissue slices may be cultured so that they are not attached to a substrate and then placed between a first and second support members. Several slices can be stacked in this fashion.

### EXAMPLE 1

### MRI of Cultured Rat Hippocampal Slices

In this example, the inventors describe method embodiment that provides high-quality MRI data from cultured rat hippocampal slices imaged using a 14.2-T magnet. Slice cultures share the advantages of acutely-prepared brain slices, but are thin enough for correlative microscopy techniques, remain viable for several weeks and do not require perfusion for some imaging studies even at 35° C. Because slice cultures can remain viable for weeks instead of hours, slice cultures may enable MRI investigations of stem cell transplantation and migration, or MRI characterizations of chronic perturbations to tissue microstructure (e.g. Alzhemier's-like plaques)(10, 11). The equilibrated and stable biochemistry of cultured brain slices also suggests this method may provide a better experimental platform for the development of molecular imaging technologies (e.g. imaging of gene expression)(12).

Culture of Rat Hippocampal Slices

When hippocampal slice cultures are prepared from young rodents, the neural connections of the hippocampal formation mature and maintain a 3-dimensional, organotypic organization for several weeks in vitro (13, 9). The particular methods used to culture hippocampal slices for this study were described previously (14, 15). Briefly, 375-μm slices were cut horizontally from the brain of P9 Wistar rats (Charles River, Wilmington, Mass.) using a vibratome (VS1000, Leica, Bannockburn, Ill.). Slices that contained dentate gyrus, entorhinal cortex and adjacent temporal cortex were cultured under interphase conditions in a humidified 5% CO₂ atmosphere at 35° C. (16). For the first 5 days, slices were cultured in horse serum-containing medium increasingly replaced by serum-free, defined solution based on DMEM-FI2 with N2 and B27 supplements. Under these conditions, field excitatory post-synaptic potentials could be recorded in the perforant and Schaffer collateral pathways up to 33 days in culture, suggesting that the functional integrity of the slice culture preparation was preserved. More than 75% of all the hippocampal slices cultured under these conditions retain in vivo hippocampal cytoarchitecture (i.e. preservation of the major subpopulations without mossy fiber sprouting and only mild gliosis)(15). After 7 days of culture, slices with abundant gliosis or visible neuronal loss by light microscopy were discarded.

Preparation of Slice Cultures for MRI

After 2-weeks incubation, individual rat hippocampal slice cultures were carefully cut from the culture membranes with a scalpel and fine surgical scissors such that the outer edges of the remaining membrane with attached slice culture approximated the inner diameter of a 10-mm NMR tube (for example one as shown in FIG. 1). A bottom Delrin anchor with attached hollow tubing was then immersed into a 10-mm NMR tube filled with fresh slice culture media. For these experiments, this tubing was solely used to aid recovery of the slices after MRI experiment completion, but the tubing could also be used for inflow perfusion if required (2).

Two 200-micron thick Delrin rings were designed to compress the outer edges of the cut slice culture membrane (such as that discussed above in relation to FIG. 1,). Both Delrin rings had a 9-mm outer diameter to fit inside a 10-mm outer-diameter glass NMR tube and a notch cut to allow passage of fluid and the tubing attached to the bottom Delrin anchor. The bottom ring had a narrow inner diameter (4 mm) to provide additional support for the slice culture membrane that rests on top of it while the top ring had an 8-mm inner diameter to prevent compression of the slice culture. Working just below the meniscus of the culture media in the NMR tube, the bottom ring is placed into the NMR tube, followed by the culture membrane with attached slice culture and then the thinner top ring is added to press the culture membrane flat and horizontal. With this embodiment, it is critical that the tissue-covered surface of the slice culture membrane point upwards to avoid compressing the cultured slice against the thicker bottom ring. If the tissue surface of the cut culture membrane insert was apposed to the bottom bracket, the tissue would be crushed and nonviable. This step can be repeated such that as many as 20-30 slice cultures can be imaged simultaneously. A top Delrin piece was then placed over the assembly to protect the top slice cultures from contact and to maintain consistent compression on

The resultant assembly was then lowered to the bottom of the 10-mm NMR tube with a glass rod. If desired, this glass rod can be secured in place to maintain excellent compression on the rings holding the slice culture membranes in horizontal position. Additional outflow lines then can be added to withdraw perfusate if continuous perfusion of the slice cultures were required during the MRI experiment (2). The culture media above the slice cultures can also be gassed with 95% O₂/5% CO₂ to prolong slice viability by maintaining high oxygen tensions and buffering physiological pH in the media. The tube was then tightly sealed with a slotted NMR-tube cap and parafilm. This assembly process must occur under sterile conditions to avoid infection if live slice cultures are to be used for experiments longer than 10-12 hrs (e.g. time-intensive MRI studies, subsequent correlative microscopy after MRI data collection, or re-incubation of cultures after MRI data collection). Autoclave sterilization may distort the fine tolerances of the Delrin pieces so it is preferable to treat the equipment with ethylene oxide gas for sterilization.

MRI of Slice Cultures

MRI data were obtained at room temperature using a 10-mm birdcage coil interfaced to a Bruker 14.1-T vertical magnet and console with 3000 mT/m imaging gradients. Pilot multislice axial, sagittal and coronal T₁ and diffusion-weighted imaging sequences were used to locate the Delrin rings and slice cultures, then to optimize the positions of 100-μm thick axial MR-defined slices through the center of the approximately 150-μm thick rat hippocampal slice cultures. Diffusion, T₁ and T₂ measurements suitable for a two-compartment analytical model of water diffusion (see below) were collected from 11 rat hippocampal slice cultures. Additional slice cultures that showed substantial volume averaging with ACSF perfusate (N=2) or large susceptibility artifacts from air bubbles (N=2) were rejected from subsequent imaging studies and analysis.

Images for analysis with the two-compartment analytical model of tissue microstructure had limited in-plane resolution (128×64 matrix, 1.5 cm FOV) to improve the signal-to-noise ratio (SNR) while reducing the time required per scan. Water diffusion measurements in slice cultures employed a pulsed-gradient spin-echo multislice sequence with 12 diffusion-weighted images using diffusion gradients aligned with the phase gradient (0-950 mT/m) and T_{d}'S of 10, 20, 35 ms (δ=3 ms). Diffusion gradient strengths were employed so that each T_{d} measurement yielded images with b-values between 7 and 10,000 s/mm² (including imaging cross-terms). Diffusion measurements had 4 averages with a 1.5-s repetition time while echo time was minimized with respect to T_{d} (23.3, 33.3 and 48.3 ms respectively) (1.25 hours per T_{d} acquisition). T₁ values were measured in slices with a partial saturation experiment using 8 different repetition times between 125 ms and 10 s (TE=10 ms). T₂ values were determined with a multiecho sequence using 30 consecutive 10-ms echo images (TR=10 ms). The T₁ and T₂ measurements required approximately 20 and 11 minutes for completion, respectively. Signal-to-noise ratios (SNR) for diffusion-weighted images were calculated based on the mean slice culture signal minus the mean noise divided by the standard deviation of the noise.

A two-pool diffusion model with exchange (17) was fitted to the T₁, T₂ and diffusion data. This model assumes restricted diffusion in the intracellular space (that is dependent on diffusion time), extracellular water diffusion mediated by tortuosity and accounts for water exchange between the intra- and extracellular compartments of tissue. Fitting this model to the data provides estimates of the apparent diffusion coefficient (ADC) in the extracellular space, the intracellular diffusion coefficient, the average cell dimension, the mean intracellular residence time and the intracellular volume fraction for rat hippocampal slice cultures.

In addition to the above MRI studies, high-resolution diffusion-weighted and gradient echo images were collected from several additional cultured hippocampal slices to assess the laminar anatomy of the hippocampal slice cultures. Diffusion-weighted images at 78-μm in-plane resolution were acquired in 3.5 hrs (thickness=100 μm, TR/TE=1500/23.3 ms, 64 averages, T_{d}=10 ms, b=2028 s/mm²). T₂*-weighted gradient echo images of rat hippocampal slice cultures at 39-μm in-plane resolution were acquired in 25 minutes (thickness=80 μm, TR/TE=185/12.5 ms, flip angle=30°).

Assessment of Slice Culture Viability

To simulate the conditions experienced by slice cultures during the MRI experiments, additional slices were cut from their membrane inserts and immersed in culture media inside 6-well dishes. The volume of media per well was similar to the total volume of media per slice in the 10-mm NMR tubes during the MRI experiments. Thus prepared, slices were incubated either in sealed dishes at room temperature or in dishes inside a culture incubator containing 5% CO₂ at 35° C. For each group, 3 slice cultures were incubated in culture media for 2, 4, 8 or 12 hours prior to immersion-fixation with 4% formaldehyde, from paraformaldehyde, in phosphate buffer (pH 7.4). Slice cultures were visualized with phase-contrast microscopy immediately prior to fixation to assess the structural integrity of the different regions of the hippocampus and entorhinal cortex. After chemical fixation, slice cultures were processed with immunohistochemistry for glial fibrillary acidic protein (GFAP), DAPI and neuron-specific nuclear protein (NeuN) using standard published protocols (14). The resulting histological images were analyzed qualitatively relative to one another for tissue changes associated with the immersion conditions required for MRI study of slice cultures. For image interpretation, GFAP immunoreactivity was used to assess glial reaction, DAPI staining was used to assess nuclear changes and NeuN staining was used to assess neuronal soma changes.

Results

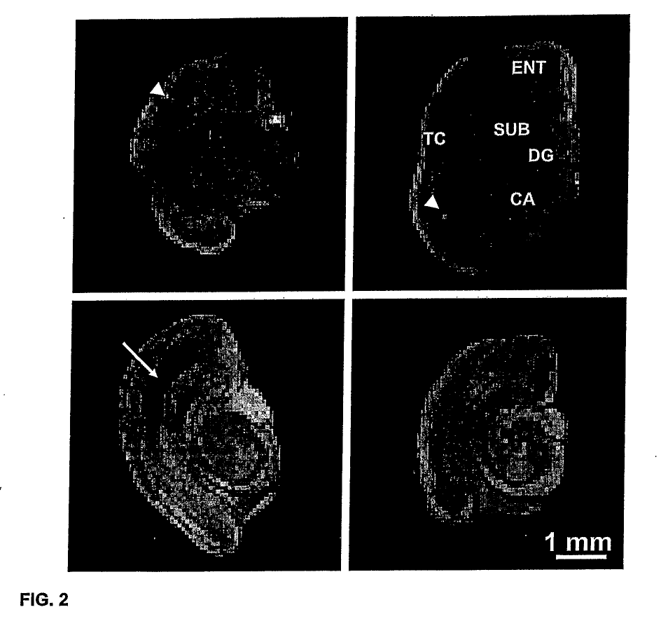

The method described in this Example 1 was simple to use and provided high quality MRI from rat hippocampal slice cultures. The laminar anatomy of the hippocampus, entorhinal and temporal cortex were well-preserved in rat hippocampal slices cultured for 2+ weeks then setup for MRI characterization (FIG. 2). A small region of subcortical white matter in the temporal cortex region atrophies during the culture process due to axonal transection during the slice procurement process. The regions of the hippocampus (e.g. CA1, CA3, dentate gyrus and subiculum) are readily discernible in diffusion or gradient echo MRI of the slice cultures (FIGS. 2 & 3). It was also possible to distinguish different cytoarchitectural layers within these regions, such as the molecular layer, granule cells and hilum of the dentate gyrus (not labeled in figures for clarity). Even with high-resolution image matrices and significant diffusion-weighting, MRI of the rat hippocampal slice cultures in a 14.1-T magnet with a 10-mm birdcage coil provided excellent SNR per unit time (e.g. 14.7:1 at b=10000 s/mm² with 2.74 nL voxels in 6 minutes)(Table 1).

Diffusion-weighted signal attenuation curves in the rat hippocampal slice cultures were non-monoexponential at all diffusion times studied. The significant dispersion of the signal attenuation curves at diffusion times of 10, 20 or 35 ms also suggests that water diffusion in the slice cultures was affected by restriction and/or exchange between 2+ unique water diffusing compartments within the tissue during the timescale of the experiment. The two-compartment model with exchange fitted the diffusion MRI data well (chi² test statistic<2) and provided estimates of the intracellular diffusion coefficient (1.46±1.51 μm²/ms), extracellular apparent diffusion coefficient (0.694±0.066 μm²/ms), apparent restriction diameter (3.35±0.50 μm), water exchange rate (69.3±14.1 s⁻¹) and intracellular water fraction (0.372±0.038, no units) [mean±SD, 11 slices]. The significant variance in the intracellular diffusion coefficient of cultured brain slices reflects the difficulty of measuring this value accurately because of restriction effects (17). The T₁ and T₂ values of slice cultures were determined to be 1.936±0.048 s and 0.064±0.005 s respectively [mean±SD, 11 slices]. The relaxation and water diffusion properties of rat hippocampal slice cultures noted above were similar to previous values described for acutely-prepared rat and human cortical brain slices (see below).

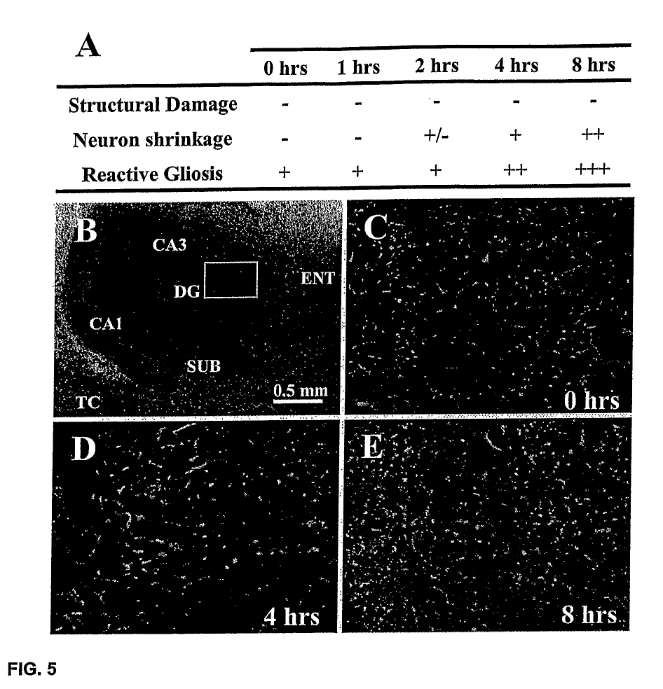

Slice cultures tolerated the immersion conditions at 20° or 35° C. required for MRI study reasonably well for 4+ hours before significant changes were observed (FIG. 5). In fact, with phase-contrast microscopy no structural pathology was observed in hippocampal slice cultures at 20° or 35° C. even after 8 hours immersion. GFAP immunoreactivity in rat hippocampal slice cultures increased significantly from background levels only after 4 hours immersion at 20° C. Similarly, NeuN staining indicated some reductions in neuronal soma sizes after 4 hours immersion at 20° C., but there were only minimal pyknotic changes or apoptotic bodies observable with DAPI staining even at 8 hours immersion. No regional differences within the rat hippocampal slice cultures for responses to increasing immersion time were observed (e.g. CA3 versus CA1). The addition of 95% O₂/5% CO₂ to the slice cultures immersed at 35° C. largely mitigated the anticipated acceleration in pathological changes such that the GFAP and NeuN changes described above also were not observed until after 4 hours immersion at 35° C. Notably, there was significant variability in GFAP immunoreactivity and neuronal size (as indicated by NeuN staining) for the individual rat hippocampal slice cultures within the same treatment groups.

Discussion

This study demonstrates the feasibility and remarkable ease of MRI investigations using cultured rat hippocampal slice cultures incubated for 2+ weeks prior to MRI investigation. Despite the inherent thinness of slice cultures (˜150 μm), diffusion MRI of slice cultures using even heavy diffusion-weighting (b˜10000 s/mm²) had reasonable SNR (>14:1) in acquisition times less than 10 min. High SNR was also readily available from less signal-attenuated MRI contrast mechanisms, such as T₁, T₂ or gradient-echo weighted images. These images provided more than adequate resolution and contrast to distinguish the different cytoarchitectural lamina and regions of the cultured rat hippocampal slices. The T₁ values of cultured slices were indistinguishable from acute brain slices, but mean T₂ values were reduced approximately 35% from 98 to 64 ms (7). This may be attributed to iron in the culture media used during the MRI acquisition for cultured slices and/or to swelling from procurement increasing the inherent T₂ of acutely-prepared brain slices.

Although from different brain regions (hippocampus versus cortex), it is interesting to compare the mean parameters obtained from the two compartment model with exchange (17) for the cultured hippocampal slices with previous model data from acute cortical brain slices (7). The mean fraction of intracellular water in cultured slices was approximately 50% smaller (0.70 to 0.37, no units). This difference could represent a decrease in overall cellular density or an increase in the astrocyte-to-neuron ratio in cultured rat hippocampal slices due to neuronal attrition during the culture process. It also may reflect the absence of finely structured neuropil development in the embryologically younger cultured slice nervous tissue (postnatal day 9). The accelerated transmembrane exchange of water in cultured hippocampal slices (67 s⁻¹) may also reflect a reduction in fine neuropil structures or it could be due to differences in membrane permeabilities. These differences also may be explained largely by the significant cytoarchitectural heterogeneity of cultured hippocampal slices (FIG. 2) compared to the acutely-prepared cortical slices. In fact, the mean intracellular diffusion coefficient and extracellular ADCs were not significantly different. This suggests that despite some differences in the intracellular fraction and exchange time that may reflect differences in brain region-specific cytoarchitecture and function, the intracellular and extracellular environments sampled by water were actually nearly identical in acutely prepared and cultured brain slices. The data in this study indicates that slice cultures offer a reasonable model of nervous tissue for MRI investigations that is more realistic than other static models of nervous tissue such as erythrocyte ghosts (18).

Besides exhibiting comparable MRI-observed tissue microstructure to rat nervous tissue, cultured rat hippocampal slices maintained excellent health under the conditions required for MRI investigation for at least 4 hours at room temperature even without perfusion or gas exchange. Further, this tolerance extended to cultured slices under magnet conditions at 35° C. when slices also were incubated with 95% O₂/5% CO₂. The changes observed with GFAP and NeuN immunohistochemistry suggested a moderate reactive gliosis and some mild neuronal shrinkage occurred in cultured slices after 4 hours under MRI-like immersion conditions. However, it was notable that there was some baseline increased GFAP immunoreactivity (from the culture process) and the observed immunohistochemistry differences from increasing immersion time were only slightly greater than the magnitude of individual cultured slice variability. Nuclear pyknosis, a finding correlated with the initiation of apoptotic cell death cascades, was a rare observation in DAPI immunohistochemistry of cultured slices after 4 or 8 hours of immersion. Also, phase-contrast microscopy did not indicate gross microstructure changes to the slice cultures even after 8 hours incubation. These findings suggest that choosing healthy rat hippocampal slice cultures prior to the MRI experiment may best determine how long the samples can tolerate immersion conditions beyond 4 hours. In contrast to these findings, our previous studies in acutely prepared rat brain slices demonstrated early evidence of microstructure damage to the infrapyramidal blade of the dentate gyrus via microscopy (6) and increased calpain activation following brain slice procurement (unpublished immunoblot data).

Cultured slices tend to be more viable and tolerate longer periods without perfusion under magnet conditions than acute rat brain slices because they are embryologically younger, have already adjusted to Wallerian degeneration initiated during tissue procurement and their inherent thinness reduces problems from delayed diffusion of nutrients into the tissue core. Data from this study indicates that cultured rat brain slices may have limitations to the total time they tolerate imaging conditions even with perfusion (similar to acute brain slices). However, the main experimental advantage of cultured rat brain slices over acute brain slices is that they can survive for 30+ days after procurement and be re-cultured after initial MRI characterizations; this feature should allow studies of more chronic pathologic processes as well as characterizing cellular processes that occur over days or weeks. Also, without the strict necessity of perfusion, this setup can be simpler than methods for MRI of acutely-prepared brain slices (2). There is also less chance of tissue movement during the MRI experiment because the slice cultures are adherent to a culture membrane that is firmly pressed between the brackets. This novel apparatus also enabled the first gradient echo images of brain slices by eliminating the polypropylene mesh (and its associated susceptibility artifacts) described in previous studies (2). Although the thinness of slice cultures creates additional challenges to obtaining sufficient SNR, conversely this thinness is more amenable to correlative techniques that may prove essential to certain MRI investigations of tissue microstructure (e.g. confocal microscopy) and facilitates higher throughput since more individual slice cultures can be stacked in the NMR tube within the sensitive region of the radiofrequency coil.

There are, however, additional challenges when using cultured brain slices to model in vivo nervous tissue for MRI investigations. Cultured brain slices differ from in vivo nervous tissue in their astrocyte-to-neuron ratio and surviving neuronal connectivity. Cultured slices gradually thin as the tissue atrophies over a period of several weeks (9), albeit that this process is significantly slower than in acute brain slices that become nonviable after only 10-12 hours. It is also important to be vigilant about sterile technique during the experiments, particularly if slice cultures are to be re-cultured after the MRI study is completed. Obtaining MRI data with sufficient SNR for complicated water diffusion experiment acquisitions within 4 hours may require imaging slice cultures at field strengths approaching 14.1 T as described here, but data from this study also suggests that useful T₁, T₂, diffusion-weighted and gradient echo contrast images can be obtained using lower magnetic field strengths (e.g. 9- or 11.7-T magnets). Further, other versions of the present design, such as a vertically-oriented Delrin compression rings inside a 5-mm NMR tube, could be used to increase sample SNR per unit time.

Similar to acutely-prepared brain slices, cultured hippocampal slices retain the 3-D cytoarchitecture of in vivo nervous tissue, tolerate long imaging protocols in high-field, narrow-bore research magnets without movement and lack a blood-brain-barrier to give experimental control over the extracellular environment. In addition, this study indicates that cultured brain slices have several additional advantages over acutely-prepared brain slices, including ease of use (without the necessity of perfusion), a better tolerance for physiological temperatures, and less swelling and acute pathology from procurement. Slice cultures can be grown for several weeks and may potentially be re-cultured after imaging, which should prove useful for MRI investigations of contrast mechanisms (e.g. diffusion) or delayed pathologic tissue reorganization. Although slice cultures are significantly thinner than acutely-prepared brain slices, this study indicates that MRI of cultured slices at medium field strengths is easily attainable. The thinness also makes slice cultures more amenable to correlative techniques such as confocal microscopy. In addition, gradient echo imaging of tissue slice cultures should enable the evaluation of molecular and intracellular contrast agents for molecular and stem cell MRI investigations. These advantages are also available to cultures of other tissue types.

### REFERENCES

1. Norris D G. The effects of microscopic tissue parameters on the diffusion weighted magnetic resonance imaging experiment. NMR Biomed 2001; 14:77-93.
2. Shepherd T M, Blackband S J, Wirth E D, III. Simultaneous diffusion MRI measurements from multiple perfused rat hippocampal slices. Magn Reson Med 2002; 48:565-569.
3. Shepherd T M, Wirth E D, III, Thelwall P E, Chen H X, Roper S N, Blackband S J. Water diffusion measurements in perfused human hippocampal slices undergoing tonicity changes. Magn Reson Med 2003; 49:856-863.
4. Buckley D L, Bui J D, Phillips M I, Zelles T, Inglis B A, Plant H D, Blackband S J. The effect of ouabain on water diffusion in the rat hippocampal slice measured by high resolution NMR imaging. Magn Reson Med 1999; 41:137-142.
5. Bui J D, Buckley D L, Phillips M I, Blackband S J. Nuclear magnetic resonance Imaging measurements of water diffusion in the perfused hippocampal slice during N-methyl-D-aspartate-induced excitotoxicity. J Neurosci 1999; 93:487-490.
6. Shepherd T M, Thelwall P E, Blackband S J, Pike B R, Hayes R L, Wirth E D, III. Diffusion magnetic resonance imaging study of a rat hippocampal slice model for acute brain injury. J Cereb Blood Flow Metab 2003; 23:1461-1470.
7. Shepherd T M, Thelwall P E, Stanisz G J, Blackband S J. Analytical Model of Water Diffusion in Isocortex Slices. Proc Intl Soc Magn Reson Med 2004; 12:1229.
8. Gahwiler B H, Thompson S M, McKinney A, Debanne D, Robertson R T. Organotypic Slice Cultures of Neural Tissue. In: Banker, Goslin K, Editors. Culturing Nerve Cells. Boston: MIT Press; 1998. p 461-499.
9. Gahwiler B H, Capogna M, Debanne D, McKinney R A, Thompson S M. Organotypic slice cultures: a technique has come of age. Trends Neurosci 1997; 20:471-477.
10. Harrigan M R, Kunkel D D, Nguyen L B, Malouf A T. Beta amyloid is neurotoxic in hippocampal slice cultures. Neurobiol Aging 1995; 16:779-789.
11. Brendza R P, Simmons K, Bales K R, Paul S M, Goldberg M P, Holtzman D M. Use of YFP to study amyloid-beta associated neurite alterations in live brain slices. Neurobiol Aging 2003; 24:1071-1077.
12. Louie A Y, Huber M M, Ahrens E T, Rothbacher U, Moats R, Jacobs R E, Fraser S E, Meade T J. In vivo visualization of gene expression using magnetic resonance imaging. Nat Biotechnol 2000; 18:321-325.
13. Zimmer J, Gahwiler B H. Cellular and connective organization of slice cultures of the rat hippocampus and fascia dentata. J Comp Neurol 1984; 228:432-446.
14. Benninger F, Beck H, Wernig M, Tucker K L, Brustle O, Scheffler B. Functional integration of embryonic stem cell-derived neurons in hippocampal slice cultures. J Neurosci 2003; 23:7075-7083.
15. Scheffler B, Schmandt T, Schroder W, Steinfarz B, Husseini L, Wellmer J, Seifert G, Karram K, Beck H, Blumcke I, Wiestler O D, Steinhauser C, Brustle O. Functional network integration of embryonic stem cell-derived astrocytes in hippocampal slice cultures. Development 2003; 130:5533-5541.
16. Stoppini L, Buchs P A, Muller D. A simple method for organotypic cultures of nervous tissue. J Neurosci Methods 1991; 37:173-182.
17. Stanisz G J, Li J G, Wright G A, Henkelman R M. Water dynamics in human blood via combined measurements of T2 relaxation and diffusion in the presence of gadolinium. Magn Reson Med 1998; 39:223-233.
18. Thelwall P E, Grant S C, Stanisz G J, Blackband S J. Human erythrocyte ghosts: exploring the origins of multiexponential water diffusion in a model biological tissue with magnetic resonance. Magn Reson Med 2002; 48:649-657.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference to the extent they are not inconsistent with the teachings herein.

What is claimed is:

1. A method of producing magnetic resonance imaging data of live tissue, said method comprising:
obtaining a tissue sample comprising live cells;
culturing said tissue sample under conditions to keep cells of said tissue sample alive for at least 12 hours; and
subjecting said tissue sample to magnetic resonance imaging.

2. The method of claim 1, wherein said obtaining step comprises making a tissue slice.

3. The method of claim 1, wherein said live tissue is central nervous system tissue, cardiac tissue, vasculature tissue, skeletal muscle tissue, gastrointestinal tissue, glandular tissue, liver tissue, spleen tissue, connective tissue, or bone tissue.

4. The method of claim 3, wherein said live tissue is brain tissue.

5. The method of claim 1, wherein said culturing step comprises culturing said tissue sample onto a substrate.

6. The method of claim 5, further comprising the securing said substrate between a top support member and a bottom support member.

7. The method of claim 6, further comprising putting said substrate with tissue sample thereon in a chamber before or during said subjecting step.

8. The method of claim 7, wherein said chamber is cylindrical.

9. The method of claim 8 wherein said chamber has an internal diameter of less than 15 mm.

10. The method of claim 8 wherein said chamber has an internal diameter of less than 12 mm.

11. The method of claim 7, wherein said chamber has a cross sectional area of no more than 1-2 mm more than the greatest diameter of the cultured tissue.

12. The method of claim 7, wherein said chamber is polygonal or arcuate, or both.

13. The method of claim 1, wherein culturing comprises culturing said tissue sample under conditions to keep cells of said tissue sample alive for at least 36 hours.

14. The method of claim 1, wherein culturing comprises culturing said tissue sample under conditions to keep cells of said tissue sample alive for at least 5 days.

15. The method of claim 1, wherein culturing comprises culturing said tissue sample under conditions to keep cells of said tissue sample alive for at least 10 days.

16. A system useful for obtaining magnetic resonance imaging data of a live tissue sample, said system comprising:
a first support member comprising a perimeter defining a first interior space;
a second support member; said first and second support members being configured such that they can secure a substrate having said live tissue sample disposed thereon when said first and second support members are brought together; and
a chamber into which said first and second support members, when brought together, may be disposed, said chamber being comprised of a material suitable for obtaining magnetic resonance imaging data from said live tissue sample when contained within said chamber.

17. The system of claim 16, wherein said chamber is cylindrical.

18. The system of claim 17, wherein said chamber has an internal diameter of 12 mm or less.

19. The system of claim 17, wherein said chamber has an internal diameter of from 11 mm to 3 mm.

20. The system of claim 16, wherein the chamber has an internal cross-sectional surface area of no more than 1-2 mm greater than the largest dimension of the cultured tissue.

21. The system of claim 16, wherein said first support member, second support member or substrate, or combination thereof, is configured to allow fluid to predominantly cross a plane of said substrate at least one location that is not proximate to a tissue when disposed on said substrate.

22. The system of claim 21, wherein said first support member, second support member or substrate, or combination thereof, are configured to define at least one portal that is not proximate to a tissue when disposed on said substrate and through which fluid may predominantly cross said plane of said substrate.

23. A tissue slice culturing scaffold comprising a substrate onto which a tissue sample may be disposed and comprising a perimeter; at least one support structure attached to or integrated with said perimeter, wherein said at least one support structure is configured to engage an inner wall of a chamber to thereby suspend said substrate in said chamber; wherein said scaffold is configured to allow fluid to predominantly cross a plane of said substrate at least one location that is not proximate to a tissue when disposed on said substrate.

24. Method of identifying MRI contrasting agents comprising
obtaining a tissue sample comprising live cells;
culturing said tissue sample under conditions to keep cells of said tissue sample alive for at least 12 hours;
exposing said tissue sample to a MRI contrasting agent candidate; and
subjecting said tissue sample to magnetic resonance imaging.

25. The method of claim 24, wherein obtaining a tissue sample comprises obtaining a tissue slice.

26. The method of claim 24, wherein said tissue sample is brain tissue.

27. The method of claim 26, wherein said tissue sample comprises histological structures corresponding to structures associated with a neuropathology and said method further comprises the step of determining whether said MRI contrasting agent candidate enhances contrasting of said histological structures.

28. The method of claim 27, wherein said histological structures are beta amyloid plaques, neurofibrillary tangles, amyloid angiopathy, and granolovacuolar degeneration.

29. The method of claim 7, wherein said tissue sample is brain tissue.

30. The method of claim 7, wherein said tissue sample is central nervous system tissue, cardiac tissue, vasculature tissue, skeletal muscle tissue, gastrointestinal tissue, glandular tissue, liver tissue, spleen tissue, connective tissue, or bone tissue.

31. The scaffold of claim 23, wherein said perimeter comprises a polygonal shape or arcuate shape, and wherein the support structure height is 5 mm or less, 2 mm or less, 1 mm or less, 500 microns or less or 250 microns or less.

32. The scaffold of claim 31 wherein said perimeter has a width of less than 15 mm at its widest dimension.

33. The scaffold of claim 31 wherein said perimeter has a width of less than 12 mm at its widest dimension.

34. The method of claim 1, wherein said tissue sample is cultured for a first culturing period; subjected to a first magnetic resonance imaging step after said first culturing period; and cultured for a second culturing period after said first magnetic resonance imaging step.

35. The method of claim 34, wherein said tissue sample is subjected to a second magnetic resonance imaging step after said second culturing period.

36. A one-piece tissue slice culturing scaffold comprising a substrate onto which a tissue sample may be disposed and comprising a perimeter; at least one support structure attached to or integrated with said perimeter, wherein said at least one support structure is configured to engage an inner wall of a chamber to thereby suspend said substrate in said chamber; wherein said scaffold is configured to allow fluid to predominantly cross a plane of said substrate at least one location that is not proximate to a tissue when disposed on said substrate.

37. The one-piece tissue slice culturing scaffold of claim 36, wherein the support structure height is 5 mm or less, 2 mm or less, 1 mm or less, 500 microns or less or 250 microns or less.

38. The one-piece tissue slice culturing scaffold of claim 36, wherein said support structure comprises at least one opening defined therein for allowing fluid to predominantly cross a plane of said substrate.

39. The one-piece tissue slice culturing scaffold of claim 36, wherein said support structure comprises openings defined contiguous to the outer surface of a support structure
